(19) United States
(12) Patent Application Publication
(10) Pub. No.: US 2007/0286815 A1
(43) Pub. Date: Dec. 13, 2007

(54) Medicinal Aerosol Formulations and Methods of Synthesizing Ingredients Therefor

(76) Inventors: Kevin J. Bechtold, Mound, MN (US); John T. Capecchi, Oakdale, MN (US); Robert S. Davidson, Edina, MN (US); Sarah B. Gunderson, Cincinnati, OH (US)

Correspondence Address: 3M INNOVATIVE PROPERTIES COMPANY, PO BOX 33427, ST. PAUL, MN, 55133-3427, US

(21) Appl. No.: 11/575,941

(22) PCT Filed: Sep. 6, 2005
(86) PCT No.: PCT/US05/31716

§ 371 Date: Mar. 23, 2007

Related U.S. Application Data
(60) Provisional application No. 60/613,063, filed on Sep. 24, 2004.

(51) Int. Cl. A61K 9/12; A61M 11/00

(52) U.S. Cl. 424/45; 128/200.14

(57) ABSTRACT

A medicinal aerosol product and process, such as for an HFA metered dose inhaler, wherein at least one of the formulation ingredients is synthesized by running at least part of the chemical reaction to make the ingredient in a hydrofluoroalkane reaction medium.

The present invention relates to medicinal aerosol products and methods of synthesizing compounds for inclusion in medicinal aerosol formulations. This application claims priority to U.S. Provisional Patent Application Ser. No. 60/613,063, filed on Sep. 24, 2004, which is incorporated herein by reference.

### BACKGROUND OF THE INVENTION

Solution-based chemical reactions are typically performed using water and/or conventional organic solvents as a reaction medium. Although there is a wealth of information on performing such reactions, it can oftentimes be difficult to effectively remove residual reaction solvent from the end product of the reaction. Presence of water and/or conventional organic solvents can often be detrimental to a finished product composition. For example, in pharmaceutical compositions the presence of water can leas to degradation of the active pharmaceutical ingredient or physical instability, such as recrystallization of amorphous compounds. In addition, the presence of conventional organic solvents can present a toxicological concern requiring precise control of residual solvents to very low levels. It is thus desirable to be able to prepare materials for use in certain applications by a synthetic method that does not rely upon water and/or conventional organic solvents as a reaction medium.

### SUMMARY

It has now been found that medicinal aerosol formulations can be made having reduced levels of water and/or other impurities by synthesizing one or more of the composition ingredients, such as drug, oligolactic acid polymers, polyethylene glycols, polyvinylpyrrolidones, or other ingredients, in a hydrofluoroalkane reaction medium. This can be particularly beneficial where the ingredient is to be in a formulation of the same hydrofluoroalkane compound, such as BFA-134a and/or 227, used in the medicinal aerosol formulation.

One embodiment provides a method of making a medicinal aerosol product, comprising filling into a medicinal aerosol product container a drug-containing formulation suitable for aerosolization, wherein at least one compound included in such formulation is synthesized by steps including providing a reaction medium including a hydrofluoroalkane; combining the reaction medium with one or more reactants; and allowing a chemical reaction to proceed thereby forming a reaction product. The medicinal aerosol product may be, for example, a metered dose inhaler for oral or nasal inhalation. The invention may also be used in the context of other medicinal aerosol products and formulations, such as dry powder inhalers and nebulizers. In one embodiment, when the medicinal aerosol is a pressurized metered dose inhaler, it includes as propellant HFA-134a and/or HFA-227.

Medicinal aerosols are often used to deliver drugs for the treatment of asthma, allergy, or chronic obstructive pulmonary disease, but other drugs may also be used, including drugs for systemic delivery.

The reaction medium is a hydrofluoroalkane, for example HFA-134a and/or HFA-227. In one embodiment the reaction product made by reacting one or more reactants in a reaction medium including a hydrofluoroalkane is hydrofluoroalkane-soluble.

In one embodiment, the present invention provides a method for preparing a reaction product. The method comprises providing a reaction medium comprising HFA-134a, wherein the reaction medium is substantially free of water. The reaction medium is combined with one or more reactants and a chemical reaction is allowed to proceed thereby forming a hydrofluoroalkane-soluble reaction product. In one aspect, the reaction product is selected from the group consisting of oligolactic acids, polyethylene glycols, and functionalized derivatives thereof.

In one embodiment, the present invention provides a method for preparing a reaction product. The method comprises providing a reaction medium comprising a hydrofluoroalkane, wherein the reaction medium is substantially free of water. The reaction medium is combined with one or more reactants and a chemical reaction is allowed to proceed thereby forming a non-fluorinated hydrofluoroalkane-soluble reaction product. In one aspect, the reaction product is selected from the group consisting of oligolactic acids, polyethylene glycols, and functionalized derivatives thereof.

In one embodiment, the present invention provides a method for preparing a pharmaceutical composition. The method comprises providing a reaction medium comprising a hydrofluoroalkane. The reaction medium is combined with one or more reactants and a chemical reaction is allowed to proceed thereby forming a reaction product selected from the group consisting of active pharmaceutical ingredients and pharmaceutically acceptable excipients. The reaction product is optionally combined in a container with an active pharmaceutical ingredient not prepared by said chemical reaction, and optionally a hydrofluoroalkane to provide a container comprising a composition comprising at least one pharmaceutical active and a hydrofluoroalkane.

### DETAILED DESCRIPTION

The reaction medium is a relatively inert compound or mixture of compounds that serves the purpose of dissolving and/or dispersing the reactants, thereby allowing for a desired chemical reaction to take place. In a preferred aspect, the reaction medium is substantially inert to chemical reaction with any of the reactants. By substantially inert, it is meant that less than 1.0%, preferably less than 0.1%, more preferably less than 0.05%, and most preferably less than 0.01% of the reaction medium will undergo chemical reaction with the reactants at a given process condition.

In one aspect, the reaction medium comprises a hydrofluoroalkane solvent. Suitable examples of hydrofluoroalkane solvents include HFA-134a, HFA-227, and fluoroform. HFA-134a and HFA-227 are preferred hydrofluoroalkanes. HFA-134a is a particularly preferred hydrofluoroalkane. In one aspect, the reaction medium is substantially free of components that are liquid when held at ambient pressure (i.e., 1 atmosphere) and at ambient temperature (i.e., 20° C.). In another aspect, the reaction medium may comprise a mixture of hydrofluoroalkane with water and/or conventional organic solvents. By “conventional organic solvents” it is understood that this describes typical organic solvents used for chemical synthesis, such as methylene chloride, toluene, ethyl acetate, methanol, ethanol, and the like, as opposed to the hydrofluoroalkanes of the present invention.

In one aspect, the reaction medium is free of conventional organic solvents. In one aspect, the reaction medium is free of water. It should be understood that in certain instances a hydrofluoroalkane reaction medium may contain trace amounts of compounds, such as conventional organic solvents or water. For example, trace amounts of compounds may be present as an impurity formed during preparation of the hydrofluoroalkane or trace amounts of compounds may form or otherwise mix with the hydrofluoroalkane during storage and prior to use as a reaction medium. Therefore, in one aspect, the reaction medium is substantially free of conventional organic solvents. By substantially free, it should be understood that the reaction medium is not an intentional blend of hydrofluoroalkane with a conventional organic solvent, although a trace amount of conventional organic solvent may be detectable. For purposes of this patent, “substantially free” indicates that the reaction medium contains less than about 0.1% by weight of a particular compound (e.g., conventional organic solvent). In one aspect, the reaction medium is substantially free of water. In one aspect, the reaction medium consists essentially of one or more hydrofluoroalkane compounds.

The reaction medium may be a gas, liquid, mixture of gas and liquid, or a supercritical fluid. In one aspect the reaction medium is a liquid. In one aspect the reaction medium is a liquid held under elevated pressure. In one aspect, the reaction medium is held under a pressure equal to the vapor pressure of the reaction medium at the temperature of the reaction. Typical pressures are between about 10 psi (69 kPa) and about 200 psi (1.4 MPa), more preferably between about 25 psi (172 kPa) and about 100 psi (690 kPa).

In methods according to the present invention, the reaction medium is combined with one or more reactants. The reaction medium and reactants are typically combined in a closed reaction vessel. This vessel is typically constructed of a relatively inert compound, such as glass or stainless steel. In one aspect, the reaction medium is added to the reaction vessel prior to addition of the reactants to the reaction vessel. In one aspect, the reaction medium is added to the reaction vessel after addition of the reactants to the reaction vessel. In one aspect, one or more reactants is added to the reaction vessel, followed by addition of the reaction medium to the reaction vessel, followed by addition of one or more additional reactants to the reaction vessel. It may be desirable to add reactants and/or reaction medium to the reaction vessel in stepped amounts, such that several individual additions are made in order to control the rate of mixing or reaction. In one embodiment, a first reactant is mixed with a portion of hydrofluoroalkane reaction medium in the reaction vessel. A second reactant is mixed with a portion of hydrofluoroalkane reaction medium in a separate vessel and added to the mixture of first reactant and hydrofluoroalkane to initiate a chemical reaction.

A reactant is a compound that can be dissolved or dispersed in the hydrofluoroalkane reaction medium, and which undergoes a chemical reaction changing covalent bonds to form a new molecule or chain of molecules (i.e., a reaction product). Hence, the reactant compounds will normally have functional groups or characteristics that allow them to undergo chemical reaction with each other or with other reactants. The reactants are preferably substantially inert with respect to the reaction medium. Also, the reaction product may in some cases be subsequently modified by further chemical reactions outside of the hydrofluoroalkane reaction medium. Non-limiting examples of suitable reactants include molecules with functional groups selected from the group consisting of carboxy, sulfonamide, urea, carbamate, carboxamide, hydroxy, amino, oxy, oxo, cyano, nitro, nitroso. Molecules with double and/or triple bonds are also examples of suitable reactants. In one embodiment, the reactant may be selected such that it reacts with like molecules to form repeating chains (i.e., oligomerization or polymerization). Such a reaction may proceed in the presence of a single reactant. It should also be noted, for avoidance of doubt, that the reaction product refers to an intended ingredient in the formulation and not merely a degradation product or the like.

In another embodiment there are at least two different reactants. One example of such a reaction is a free-radical polymerization wherein one reactant is an initiator and another reactant is a monomer that polymerizes upon initiation by the initiator. Another example of such a reaction is a condensation polymerization wherein two different reactants combine with each other to form a polymer. In one embodiment, reactants may be selected such that they initiate a polymerization reaction and are consumed during the reaction. However, catalysts that merely facilitate a reaction, but which are not consumed or chemically altered are not considered reactants for purposes of the present invention. In one embodiment, reactants may be selected so as to form an end-group on an otherwise already formed oligomer or polymer.

In one aspect, at least one of the reactants is an oligomer (i.e., 3 or more repeat units) or polymer, preferably with a reactive end-group. Examples of suitable oligomers or polymers include esters and ethers, such as those described in U.S. Pat. No. 5,569,450 (Duan et al.), U.S. Pat. No. 6,126,919 (Stefely et al.) and pending U.S. patent application Ser. No. 60/533172 (Capecchi et al.), the disclosures of which are incorporated by reference. In a preferred embodiment, one reactant is an oligomer or polymer having a reactive end-group and at least one other reactant is not an oligomer or polymer, that is, having either a single repeat unit (i.e., monomer) or two repeat units (i.e., a dimer).

Preferred oligomeric or polymeric reactants include oligolactic acids, polylactic acids, polyethylene glycols, and polyvinylpyrrolidones. More preferred reactants include oligolactic acids and polyethylene glycols. Particularly preferred reactants are oligolactic acids.

The reactants may be provided as gases, liquids, or solids, preferably as liquids or solids. In one aspect, one or more reactants may be provided to the reaction vessel at elevated temperatures. In one aspect, one or more reactants may be provided to the reaction vessel at elevated pressures. In one aspect, one or more reactants may be provided to the reaction vessel at ambient temperature

After mixing of suitable reactants in the reaction medium, a chemical reaction is allowed to proceed forming a reaction product. For purposes of the present invention, a chemical reaction indicates that one or more reactants undergo a change to one or more covalent chemical bonds. In one aspect, the chemical reaction will take place upon mixing of the reactants without further measures being taken. In one aspect, it may be necessary to heat the reactants or to add an additional reactant, such as an initiator, to initiate the reaction. In some instances it may be desirable to either heat or cool the reactants to accelerate or slow the rate of reaction. Suitable elevated temperatures are above about 30° C. In one aspect, elevated temperatures are above about 80° C.

The reaction is allowed to continue until measurable amounts of reaction product are formed, preferably allowed to continue until the reaction is more than 50% complete, more preferably allowed to continue until the reaction is more than 90% complete, and most preferably allowed to continue until substantially complete. By complete, it is understood that a given set of reactants will eventually reach a chemical equilibrium where no more reaction will occur. This is not necessarily equivalent to complete consumption of the initial reactants. For example, one or more reactants may be provided in a non-stoichiometric amount, such that residual reactant remains upon completion of the reaction. Alternatively, chemical equilibrium may be reached at a particular balance of reaction products and remaining reactants. The percentage of reaction completion is defined as the percentage of reaction product formed as a mole fraction of the amount of reaction product that would have been formed had the reaction proceeded to chemical equilibrium. In a preferred embodiment, the reactants are provided in stoichiometric amounts. In a preferred embodiment, stoichiometric amounts of the reactants are substantially consumed at chemical equilibrium.

In one aspect, the reaction product is hydrofluoroalkane-soluble (i.e., at least partially soluble in hydrofluoroalkane). In one aspect, the reaction product is essentially entirely hydrofluoroalkane soluble, and preferably entirely hydrofluoroalkane soluble. In one aspect, the reaction product is an oligomer or polymer, preferably a non-crosslinked oligomer or polymer. In one aspect, the reaction product is non-fluorinated and hydrofluoroalkane-soluble.

Preferred reaction products include oligolactic acids, polylactic acids, polyethylene glycols, polyvinylpyrrolidones, and functionalized derivatives thereof. More preferred reaction products are oligolactic acids, polyethylene glycols, and functionalized derivatives thereof. Particularly preferred reaction products are oligolactic acids and functionalized derivatives thereof.

The reaction product may be isolated from the reaction medium or alternatively, the reaction product and hydrofluoroalkane may be further acted upon as an intermediate composition (i.e., a “reaction product-hydrofluoroalkane composition”).

It may be desirable to purify the reaction product-hydrofluoroalkane composition. This may be done, for instance, by an aqueous extraction of the reaction product-hydrofluoroalkane composition. In one aspect, impurities or residual reactants from the reaction medium are extracted with an extraction solution comprising an acidic or basic aqueous solution. This may be particularly beneficial for reactant products having surfactant-like properties, as aqueous extraction of such a reactant product in a conventional organic solvent may lead to an emulsion that is not easily separated. This is also beneficial for reactant products or impurities having ionizable functional groups. The presence of ionizable functional groups often makes extraction in traditional organic solvents difficult due to the formation of stable emulsions. For instance, extraction of acidic impurities when performed in conventional organic solvent using a basic solution may lead to an emulsion if the reaction product or residual reactants also has acidic functionality. In one aspect, the present invention includes basic extraction of a reaction product-hydrofluoroalkane composition having residual reactants with acidic functionality. In one aspect, wherein the reaction product is selected from the group consisting of oligolactic acids and functionalized derivatives, the present invention includes extraction with a basic aqueous extraction solution.

In one aspect, the reaction product is isolated from the reaction medium. In one aspect, where the reaction medium is substantially free of components that are liquid at ambient temperature and pressure, the reaction product may be isolated by evaporation of the hydrofluoroalkane at ambient temperature and pressure. Addition of vacuum may be used to increase the rate and efficiency of evaporation. The reaction product may be separated from the reaction medium by precipitation or crystallization and filtration. Alternatively, the reaction product may be isolated from the reaction medium by conventional spray drying methods. A combination of one or more of the aforementioned techniques may also be used to isolate the reaction product. Other suitable isolation methods include adsorption, such as adsorption onto ion-exchange beads or other solids; absorption, such as absorption of a volatile product from the mixed vapors; distillation from the reaction medium; or liquid-liquid extraction from the reaction medium.

The isolated reaction product may be a liquid, gas, or solid. The isolated reaction product is preferably a solid, and may be in any conventional solid form, such as a powder, flake, crumb, pellet, or amorphous mass. The isolated reaction product may be crystalline, amorphous, or a mixture of crystalline and amorphous. In one aspect, the reaction product has more amorphous character than crystalline character, and preferably, the reaction product is substantially entirely amorphous.

In a preferred embodiment, the amount of impurities in the isolated reaction product is less than about 10% by weight, preferably less than about 5% by weight, more preferably less than about 1% by weight, and most preferably less than about 0.5% by weight. In one aspect, the isolated reaction product is substantially free of conventional organic solvents. In one aspect, the isolated reaction product is substantially free of water. In one aspect, the amount of residual hydrofluoroalkane is less than about 5% by weight, preferably less than about 1% by weight, more preferably less than about 0.1% by weight, and most preferably less than about 0.01% by weight. In one aspect the reaction product is substantially free of residual hydrofluoroalkane.

The isolated reaction product is particularly suited for use in medicinal aerosol products, either as a pure substance, in a mixture with other substances, or as an intermediate used for preparing a final product for use. Reaction products of the present invention find particular utility in applications where compounds of high purity are desired or in applications where compounds free from water and/or conventional organic solvents are desired. The reaction product is filled into a medicinal aerosol product container to provide a drug-containing formulation suitable for aerosolization. Suitable medicinal aerosol products include metered dose inhalers, dry powder inhalers, and nebulizers.

In one aspect, the container is a canister that may be equipped with a valve and used to contain a pressurized aerosol formulation. Addition of the reaction medium and/or hydrofluoroalkane to the container may be done under pressurized conditions. Alternatively, the reaction medium and/or hydrofluoroalkane may be chilled to a liquid condition prior to addition to the container. Examples of suitable pressurized aerosol devices useful with methods of the present invention include metered dose inhalers described in U.S. Pat. No. 4,664,107 (Wass); U.S. Pat. No. 4,819,834 (Thiel), U.S. Pat. No. 5,772,085 (Bryant et al.), U.S. Pat. No. 5,836,299 (Kwon), and U.S. Pat. No. 6,650,805 (Castro et al.), the disclosures of which are hereby incorporated by reference.

In one embodiment, the reaction product is an active pharmaceutical ingredient. In one aspect, if the reaction product is isolated from the reaction medium, then a hydrofluoroalkane may also added to the container to provide a pressurized formulation. In a second aspect, the reaction product is not isolated from the reaction medium prior to addition to the container. Additional hydrofluoroalkane may be optionally added to the container in this second aspect. A container comprising a composition comprising at least one pharmaceutical active and a hydrofluoroalkane is thus prepared.

As used herein, the term “drug,” includes its equivalents, “bioactive agent,” and “medicament” and is intended to have its broadest meaning as including substances intended for use in the diagnosis, cure, mitigation, treatment or prevention of disease, or to affect the structure or function of the body. The drugs can be neutral or ionic. Preferably, they are suitable for oral and/or nasal inhalation. Delivery to the respiratory tract and/or lung, in order to effect bronchodilation and to treat conditions such as asthma and chronic obstructive pulmonary disease, is preferably by oral inhalation. Alternatively, to treat conditions such as rhinitis or allergic rhinitis, delivery is preferably by nasal inhalation. Preferred drugs are asthma, allergy, or chronic obstructive pulmonary disease medications.

Suitable drugs include, for example, antiallergics, anticancer agents, antifungals, antineoplastic agents, analgesics, bronchodilators, antihistamines, antiviral agents, antitussives, anginal preparations, antibiotics, anti-inflammatories, immunomodulators, 5-lipoxygenase inhibitors, leukotriene antagonists, phospholipase A₂ inhibitors, phosphodiesterase IV inhibitors, peptides, proteins, steroids, and vaccine preparations. A group of preferred drugs include adrenaline, albuterol, atropine, beclomethasone dipropionate, budesonide, butixocort propionate, clemastine, cromolyn, epinephrine, ephedrine, fentanyl, flunisolide, fluticasone, formoterol, ipratropium bromide, isoproterenol, lidocaine, morphine, nedocromil, pentamidine isoethionate, pirbuterol, prednisolone, salmeterol, terbutaline, tetracycline, 4-amino-α,α,2-trimethyl-1H-imidazo[4,5-c]quinoline-1-ethanol, 2,5-diethyl-10-oxo-1,2,4-triazolo[1,5-c]pyrimido[5,4-b][1,4]thiazine, 1-(1-ethylpropyl)-1-hydroxy-3-phenylurea, and pharmaceutically acceptable salts and solvates thereof, and mixtures thereof. Particularly preferred drugs include pirbuterol, 4-amino-α,α,2-trimethyl-1H-imidazo[4,5-c]quinoline-1-ethanol, 2,5-diethyl-10-oxo-1,2,4-triazolo[1,5-c]pyrimido[5,4-b][1,4]thiazine, 1-(1-ethylpropyl)-1-hydroxy-3-phenylurea, and pharmaceutically acceptable salts and solvates thereof, and mixtures thereof.

In one embodiment, the invention comprises a method for preparing a pharmaceutical composition whereby the chemical reaction forms a reaction product selected from the group consisting of active pharmaceutical ingredients and pharmaceutically acceptable excipients. The reaction product may be isolated following the general procedures as discussed above. The reaction product may be combined with an active pharmaceutical ingredient not prepared by the chemical reaction of the present invention to provide a pharmaceutical composition comprising at least one active pharmaceutical ingredient.

In one embodiment, the reaction product is an active pharmaceutical ingredient. This may be combined with other ingredients, such as pharmaceutically acceptable excipients, and/or other active pharmaceutical ingredients, to form a pharmaceutical composition comprising at least one active pharmaceutical ingredient. Alternatively, the active pharmaceutical ingredient reaction product may be isolated to directly prepare a pharmaceutical composition, that is, a single active pharmaceutical ingredient.

As defined herein, an active pharmaceutical ingredient is any component of a drug product intended to furnish pharmacological activity or other direct effect in the diagnosis, cure, mitigation, treatment, or prevention of disease, or to affect the structure or any function of the body of humans or other animals.

In one embodiment, the reaction product is a pharmaceutically acceptable excipient. This may be combined with other ingredients, such as other pharmaceutically acceptable excipients, and/or active pharmaceutical ingredients, to form a pharmaceutical composition comprising at least one active pharmaceutical ingredient. Alternatively, the pharmaceutically acceptable excipient reaction product may be isolated directly and stored for future use.

As defined herein, a pharmaceutically acceptable excipient is an inactive component of a drug product. A pharmaceutically acceptable excipient is selected such that it has an acceptable safety profile at the concentration and/or amount employed in a drug product for a given route of administration. Pharmaceutically acceptable excipients may be used for a wide variety of purposes in pharmaceutical dosage forms. Examples of excipients include carriers, diluents, coatings, coloring agents, flavoring agents, solubilizers, stabilizers, anti-oxidants, propellants, absorption enhancers, penetration enhancers, surfactants, complexing agents, and the like.

Solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Excipients used in topical or transdermal dosage compositions include adhesive carriers, such as acrylate, silicone, and polyisobutylene polymers; excipients used to prepare gels and creams; skin penetration enhancers (i.e., substances that increase the permeation rate a drug across or into the skin) or solubilizers (i.e., substances that effectively solubilize a drug). Exemplary materials include C₈-C₂₀ fatty acids such as isostearic acid, octanoic acid, and oleic acid; C₈-C₂₀ fatty alcohols such as oleyl alcohol and lauryl alcohol; lower alkyl esters of C₈-C₂₀ fatty acids such as ethyl oleate, isopropyl myristate, butyl stearate, and methyl laurate; di(lower) alkyl esters of C₆-C₈ diacids such as diisopropyl adipate; monoglycerides of C₈-C₂₀ fatty acids such as glyceryl monolaurate; tetraglycol (tetrahydrofurfuryl alcohol polyethylene glycol ether); tetraethylene glycol (ethanol,2,2′-(oxybis(ethylenoxy))diglycol); C₆-C₂₀ alkyl pyrrolidone carboxylates; polyethylene glycol; propylene glycol; 2-(2-ethoxyethoxy)ethanol; diethylene glycol monomethyl ether; N,N-dimethyldodecylamine-N-oxide and combinations of the foregoing. Alkylaryl ethers of polyethylene oxide, polyethylene oxide monomethyl ethers, polyethylene oxide dimethyl ethers, glycerol, and N-methyl pyrrolidone are also suitable. The terpenes are another useful class of pharmaceutical excipients, including pinene, d-limonene, carene, terpineol, terpinen-4-ol, carveol, carvone, pulegone, piperitone, menthone, menthol, neomenthol, thymol, camphor, borneol, citral, ionone, and cineole, alone or in any combination.

Excipients used in aerosol dosage forms include propellants, such as HFA-134a, HFA-227, dimethyl ether, pentane; cosolvents, such as ethanol and isopropanol; lubricants, such as silicone oil; surfactants, such as oleic acid, sorbitan trioleate, and sorbitan monooleate; and taste masking ingredients, such as menthol. Other suitable propellants, cosolvents, and surfactants are disclosed, for example, in U.S. Pat. No. 5,225,183 (Purewal et al.), the disclosure of which is incorporated by reference.

Excipients used in liquid pharmaceutical compositions can include a carrier, such as, for example, water, saline, aqueous dextrose, glycerol, ethanol and the like, to thereby form a solution or suspension. Other excipients include wetting or emulsifying agents, pH buffering agents, antioxidants, and the like, such as, for example, citric acid, sorbitan monolaurate, triethanolamine oleate, and butylated hydroxytoluene may also be used.

In one embodiment, the reaction product is a pharmaceutically acceptable excipient. The reaction product and an active pharmaceutical ingredient are added to a container. In one aspect, if the reaction product is isolated from the reaction medium, then a hydrofluoroalkane is also added to the container. In a second aspect, the reaction product is not isolated from the reaction medium prior to addition to the container. Additional hydrofluoroalkane may be optionally added to the container in this second aspect. A container comprising a composition comprising at least one pharmaceutical active and a hydrofluoroalkane is thus prepared.

### EXAMPLES

### Example 1

Acetyloligolactic acid (Mₙ=643.8) was prepared according to the general methods described in U.S. patent application Ser. No. 60/533172 (Capecchi et al.). Number average molecular weight, Mₙ, was calculated from the measured degree of oligomerization, n, experimentally determined by NMR. The acetyloligolactic acid was heated in an oven at 100° C. until molten and 255.7 g (0.40 mol) was poured into a 1-gallon stainless steel pressure vessel. To this was added 61.2 g (0.38 mol, 0.95 equiv.) of 1,1′-Carbonylbis-1H-Imidazole and a stir bar. The vessel was sealed and charged with 2.5 kg HFA-134a by transferring liquid HFA-134a to the vessel using the liquid line of an HFA-134a tank. If the transfer of liquid stopped due to a buildup of backpressure before the desired amount was transferred, then a vent was opened to evaporatively cool the receiving vessel to reduce the backpressure. Once the desired amount of HFA-134a was transferred, the contents were stirred for 20 hours while being held at ambient temperature and the vapor pressure of HFA-134a, thus preparing an activated acetyloligolactic acid solution. A second pressure vessel was charged with 10.7 g (0.18 mol, 0.90 equiv.) of ethylenediamine and pressurized with 0.5 kg of HFA-134a. Nitrogen was used to bring the pressure of the second vessel to a pressure about 3 to 5 psi above that of the first vessel. The second vessel was emptied by transferring the HFA-134a solution to the first vessel using a high-pressure rated tube. The second vessel was rinsed with a charge of 0.2 kg HFA-134a and this charge was also transferred to the first vessel. The solution in the first, or ‘reaction’, vessel was then stirred for 20 hours.

The empty pressure vessel was then used as an extraction vessel by charging with 500 mL of 2.0 molal acetic acid (aq) and pressurizing with HFA-134a vapor and nitrogen as described above. The reaction vessel was emptied by transferring the HFA-134a solution from the reaction vessel to the extraction vessel. The contents of the extraction vessel were stirred for 1 hour and then allowed to rest for 30 minutes. The HFA-134a phase was then transferred to the empty vessel using high pressure tubing, and the aqueous phase discarded. The HFA-134a solution was extracted in this fashion two more times.

Next, the HFA-134a solution was extracted in similar fashion with three 400 ml portions of 50% saturated NaHCO₃/1.25 molal NaCl (aq) solution. The HFA-134a phase was then transferred to a vessel containing 100 g MgSO₄ and stirred for 4 hours. The solution was then filtered into a clean, dry pressure vessel through a high-pressure rated filter housing (Millipore Co.) equipped with a paper filter (Whattmann No. 5) to remove the magnesium sulfate. The solution was then further dried by circulating over a column containing 3Å molecular sieves for 72 hours using a diaphragm pump. The resulting solution was 10% N,N′-ethylenebis (acetyloligolactyl) amide in dry HFA-134a. The solution was passed through a high-pressure rated filter housing (Millipore Co.) equipped with a Whattmann No. 5 paper filter and sprayed into a flame dried glass jar under nitrogen purge. After transfer, the jar contained considerable liquid HFA-134a and the majority of this solvent was allowed to evaporate at ambient temperature and pressure. The jar was then placed under high vacuum for 24 hours, and N,N′-ethylenebis (acetyloligolactyl) amide was isolated as a dry white powder (144.71 g, 61.7% yield).

### Example 2

Acetyloligolactic acid (Mₙ=1602.2) was prepared according to the general methods described in U.S. patent application Ser. No. 60/533172 (Capecchi et al.). The acetyloligolactic acid was heated in an oven at 100° C. until molten and 98.0 g (0.061 mol) was poured into a 1-gallon stainless steel pressure vessel. To this was added 10.91 g (0.067 mol, 1.10 equiv.) of 1,1′-Carbonylbis-1H-Imidazole and a stir bar. The vessel was sealed and charged with 1.1 kg HFA-134a as described above in Example 1 and the contents stirred for 20 hours while being held at ambient temperature and the vapor pressure of HFA-134a. A second pressure vessel was charged with 12.22 g (0.067 mol, 1.10 equiv.) of sarcosine tert-butyl ester hydrochloride, pressurized with HFA 134a vapor and nitrogen as described above in Example 1 The EFA-134a solution from the first vessel was transferred to the second vessel using a high-pressure rated tube. The solution was then stirred for 96 hours. The first pressure vessel was charged with 1000 mL of 0.1 M acetic acid (aq), pressurized with EFA 134a vapor and nitrogen as described above in Example 1, and the HFA-134a solution from the second vessel was transferred back to the first vessel. The contents of the first vessel were stirred for 1 hour and then allowed to rest for 30 minutes. The HFA-134aphase was then drained back into the second vessel, and the aqueous phase discarded. The HFA-134a solution was extracted in this fashion once more. The resulting solution was approximately 10% N-(acetyloligolactyl)sarcosine-tert-butyl ester in HFA-134a.

A sample was obtained by spraying a small amount of the solution into a vial and allowing the solvent to evaporate. NMR analysis of the sample indicates that 97.1% of the acetyloligolactic acid starting material was converted to N- (acetyloligolactyl) sarcosine-tert-butyl ester, in HFA 134a

### Example 3

N,N′-ethylenebis (acetyloligolactyl) amide was prepared according to the general procedure described in Example 1 with the exception that the order of addition of the contents of the second vessel (ethylenediamine and HFA) and the contents of the first vessel (activated acetyloligolactic acid solution) was reversed. That is, the first vessel was emptied by transferring the activated acetyloligolactic acid solution to the second vessel (containing the ethylenediamine/HFA solution) using high pressure tubing. A positive pressure gradient was maintained by venting the second vessel as needed to lower the pressure by evaporative cooling.

The remainder of the reaction and extraction was performed according to Example 1. The resulting N,N′-ethylenebis (acetyloligolactyl) amide was isolated as a dry white powder with a 59.8% yield.

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

1. A method of making a medicinal aerosol product, comprising:
filling into a medicinal aerosol product container a drug-containing formulation suitable for aerosolization, wherein at least one compound included in such formulation is synthesized by steps including:
providing a reaction medium including a hydrofluoroalkane;
combining the reaction medium with one or more reactants; and
allowing a chemical reaction to proceed thereby forming a reaction product.

2. The method of claim 1, wherein the medicinal aerosol product is metered dose inhaler for oral or nasal inhalation.

3. The method of claim 2, wherein the medicinal aerosol formulation includes a hydrofluoroalkane propellant selected from the group consisting of HFA-134a and HFA-227, and mixtures thereof.

4. (canceled)

5. The method of claim 1, wherein the hydrofluoroalkane reaction medium is selected from the group consisting of HFA-134a, HFA-227, and mixtures thereof.

6. The method of claim 1, wherein the reaction medium is substantially free of water.

7. The method of claim 1, wherein there are at least two different reactants.

8. The method of claim 1, wherein the reaction product is a non-fluorinated compound.

9. The method of claim 1, wherein the reaction product is a non-crosslinked polymer.

10. The method of claim 1, wherein the reaction product is selected from the group consisting of oligolactic acids, polyethylene glycols, polyvinylpyrrolidones, and functionalized derivatives thereof.

11. The method of claim 1, wherein the reaction medium is substantially free of volatile organic solvents.

12. The method of claim 1, wherein the reaction medium is substantially free of components that are liquid at ambient temperature and pressure.

13. The method of claim 1, wherein the chemical reaction proceeds at an elevated pressure.

14. The method according to claim 1, further including the step of isolating the reaction product from the reaction medium prior to adding it to the formulation.

15. The method of claim 1, wherein the reaction product is isolated by evaporation of the reaction medium.

16. The method of claim 14, wherein there are impurities in the isolated reaction product of less than 5% by weight of the total weight of the reaction product.

17. The method according to claim 1, wherein the reaction product is not isolated from the reaction medium prior to the step of adding it to the container.

18. The method of claim 1, wherein the reaction product is hydrofluoroalkane-soluble.

19. The method of claim 1, further including the step of extracting impurities or residual reactants from the reaction medium within an extraction solution comprising an acidic or basic aqueous solution.

20. The method according to claim 19, wherein the reaction product is selected from the group consisting of oligolactic acids and functionalized derivatives thereof and the extraction solution is an basic aqueous solution.

21. The method according to claim 19, wherein the reaction product is selected from the group consisting of oligolactic acids and functionalized derivatives thereof and the extraction solution is an acidic aqueous solution.

22. A medicinal aerosol product made according to claim 1.

23. (canceled)
